(19) United States
(12) Patent Application Publication
(10) Pub. No.: US 2005/0209685 A1
(43) Pub. Date: Sep. 22, 2005

(54) Graft delivery and anchoring system

(76) Inventors: Edward G. Shifrin, Raanana (IL); Gennady Nickelshpur, Haifa (IL); Vladimir Idov, Haifa (IL)

Correspondence Address: WILLIAM J. SAPONE;COLEMAN SUDOL SAPONE P.C., 714 COLORADO AVENUE, BRIDGE PORT, CT, 06605, US

(21) Appl. No.: 10/802,262

(22) Filed: Mar. 17, 2004

(51) Int. Cl. A61F 2/06; A61B 17/068

(52) U.S. Cl. 623/1.23; 606/108; 606/200; 227/175.1

(57) ABSTRACT

The present invention relates to methods and apparatus for the repair of blood vessels, using a novel prosthetic graft within the blood vessels. The more concrete, the present invention relates to devices and methods for delivering and stapling endovascular grafts to the blood vessel wall. A subject of the present invention is a system and method for delivering and anchoring a graft of individual dimensions within a recipient's blood vessel. This system comprises: a) a graft configured as a tube from biologically compatible material the individual dimensions whereof, such as the diameter and length are defined by clinical state of the recipient and results of angiographic dimensions of its operated blood vessel, and b) a set of assembly units for assembling on its basis at least one device for delivering a graft and simultaneously securing this graft at both its ends to the blood vessel wall. The suggested set of assembly units includes: at least one proximal head and at least one distal head which serve to hold the graft in the process of its delivery within the blood vessel, and for securing its proximal and distal end to the wall of an operated blood vessel. The proximal and distal heads have a through axial hole with an actuator therein, as well as means for holding a corresponding graft end and means for securing this graft end to the blood vessel wall. Besides, the suggested set of assembly units includes at least one intermediate tube, at least one control means, at least one additional hold-down element, and at least one flexible control element.

### BACKGROUND OF THE INVENTION

1. Field of the Invention

The present invention relates to methods and apparatus for the repair of blood vessels, using a novel prosthetic graft within the blood vessels. In particular, the present invention relates to devices and methods for delivering and stapling endovascular grafts to the blood vessel wall.

2. Description of Related Art

Surgical treatment of some diseases of the circulatory system is often connected with the use of a prosthesis-artificial blood vessel which is integrated into the circulatory system to prevent from rupture or completely replace the recipient's injured vessel. Here the surgeon is faced by a problem: to provide a joint member for an artificial blood vessel which can keep enough reliability and rigidity and can be integrated naturally with the recipient's blood vessel and can decrease leakage of blood from the joint part between the recipient blood vessel and the artificial blood vessel when the artificial blood vessel and recipient blood vessel are connected with each other.

Numerous devices and methods are known for solving this problem. For example, Samuels in U.S. Pat. No. 5,423,851 “Methods and apparatus for affixing an endoluminal device to the walls of tubular structures within the body” describes a device which utilizes incremental inflation of a balloon cuff to deploy radially projecting barbs attached to the cuff within the plurality of recesses. At lower levels of cuff pressure, the engagement of the outer surface of the cuff with the tubular structure temporarily holds the device in place without tissue damage because the outer extremities of the barbs do not extend beyond the cuff's outer surface. If the device requires repositioning, then the cuff is easily deflated and moved to a new location and is again partially inflated. Once the location of the device has been found to be optimal, the cuff is fully inflated so as to deploy the barbs to permanently fix the device in place.

There are also known devices according to U.S. Pat. N. 5,522,881 “Implantable tubular prothesis having integral cuffs”, U.S. Pat. No. 5,693,088 “Intraluminal vascular graft”, U.S. Pat. No. 5,207,695 “Aortic graft, implantation device, and method for repairing aortic aneurysm”, U.S. Pat. No. 5,972,023 “Implantation device for an aortic graft method of treating aortic aneurysm” and U.S. Pat. No. 6,136,007 “Apparatus for handling tubing used in medical procedures” wherein there are also utilised cuffs, holding the prosthesis (graft or stent-graft) within the blood vessel.

A common drawback of all the aforesaid devices is that balloon cuffs are suitable mainly for temporary fixation of a prosthesis (stent-graft or graft) to the wall of a blood vessel. To permanently fix the prosthesis it is necessary to keep constant pressure in the cuffs, which renders these cuffs unreliable and complicated in use, especially when the prosthesis is inserted for a long period of time.

Baker et al. in U.S. Pat. No. 6,221,102 “Intraluminal grafting system” and Lasarus et al., in EP 0466518 “Endovascular grafting apparatus, system and method and devices for use therewith” describe a grafting system having a delivery catheter comprising a flexible elongate tubular member having proximal and distal extremities and capsule mounted on the distal extremity of the tubular member and including a graft disposed within the capsule. The graft is comprised of a tubular body configured to be secured to a blood vessel by a self-expanding attachment system. The attachment system comprises a generally sinusoidal wire frame having apices, which extend longitudinally outward from the end of the tubular body apices, which are secured within the tubular body. Both the protruding apices and the base apices are formed with helices, which bias the attachment system radially outward. The attachment system further includes a plurality of lumen piercing members that are oriented in a responsive relationship to the radially outward bias of the attachment system. Furthermore, the graft may be configured with a plurality synthetic fiber tufts secured to the outer surface of the tubular body to facilitate sealing the graft within the vessel. The graft may also include a plurality of crimps formed in the tubular body of the graft.

Systems for graft delivery and anchoring according to WO 9618361 “Vascular graft and delivery catheter” and WO 9614808 “Delivery catheter and graft for aneurysm repair” are designed likewise.

A common drawback of all these systems is that they do not provide secure fixation of a graft within a blood vessel.

Taheri in U.S. Pat. No. 4,872,874 “Method and apparatus for transarterial aortic graft insertion and implantation” describes an apparatus including a lengthy conduit for insertion through the blood vessel and having a distal end adapted to selectively expand and contract for cooperating with a preformed hole in the sidewall of the graft for binding the graft to or releasing the graft from the distal end. For securing the graft to the wall of the blood vessel, an apparatus embodiment includes an inflatable body adapted to retain the staple-like clips in a circular formation so that the pointed legs of the clips are directed generally radially outwardly of the circular formation and a mechanism for abruptly inflating the inflatable body to thereby drive the clips radially outwardly and through the wall of the tubular graft. A magnet can be used to retain the clips in condition against the inflatable body prior to being driven outwardly as aforesaid. The method of insertion includes the steps involved in implanting the tubular graft in the preselected blood vessel.

A drawback of this apparatus is as well unreliable graft fixation within a blood vessel as staple ends are not bent after they pierce the walls of the graft and blood vessel.

3. The Prior Art

Closest to the claimed invention are devices disclosed in U.S. Pat. No. 6,217,585 and U.S. patent appl. No. 2001/0032023 A1.

Houser et al. in U.S. Pat. No. 6,217,858 “Mechanical stent and graft delivery system” describe a deployment device, including a catheter, an expansion framework or cage near the catheter distal end, and a means for controlling expansion from a proximal end of the catheter. The cage is formed of a plurality of elongate strands coupled at opposite ends to the catheter and to the control device. Axial movement of the control device relative to the catheter either elongates the strands to radially collapse the cage, or axially reduces the distance between the strand ends for radial enlargement. In one embodiment the control device is a stylet contained in a lumen of the catheter and extending beyond the catheter distal end. The strands are fixed at their opposite ends to the catheter distal end and the stylet distal end. In another embodiment, the strands are coupled to proximal and distal rings along the catheter, with one of the rings being axially movable by a wire to radially expand and reduce the cage.

Herweck and Cross in U.S. pat. appl. 2001/0032023 A1 describe a tunneling device, which is implantable in a body, including a flexible covering to protect a medical from contamination during the handling and implantation of the device in the body. The tunneling device of the present invention may optionally include a rigid tunneler tip that allows for attachment of the tunneling device to a tunneler instrument prior to the implantation process.

Both devices described above provide graft delivery but do not provide fixation of the delivered graft on blood vessel walls.

An object of the present invention is to provide a device fo simultaneously solve 2 problems: delivery of a graft within a blood vessel using thechniques of minimally invasive surgery, and also secures fixation of the delivered and positioned graft to the blood vessel walls.

### SUMMARY OF THE INVENTION

A subject-matter of the present invention is a system for delivering and securing a graft of individual dimensions within a recipient's blood vessel (Graft delivery and anchoring system). This system comprises: a) a graft configured as a tube from biologically compatible material the individual dimensions whereof, in particular, diameter and length, are defined by clinical state of the recipient and angiographic measurements of its operated blood vessel, this graft has a proximal and distal ends, as well as b) a set of assembly units for assembling on the base thereof at least one device for delivering a graft of individual dimensions within the recipient's blood vessel and simultaneously anchoring this graft at both its ends on the blood vessel wall.

The suggested set of assembly units includes: at least one proximal and at least one distal heads for holding in place a graft in the course of its delivery within the blood vessel, as well as for securing its proximal and distal ends to the wall of an operated blood vessel. The proximal and distal heads have a through axial hole with an actuator located therein, as well as means for holding in place a corresponding graft end and means for securing this graft end to the blood vessel wall. Besides, the suggested set of assembly units includes at least one intermediate tube, at least one control means, at least one additional tube, as well as at least one flexible control element.

At least one intermediate tube connects together the proximal and distal heads. This tube is rigid in longitudinal direction and relatively flexible in lateral direction and has a through axial hole.

At least one control means is substantially fomed as a lever and serves to control the process of inserting the graft delivery device within the operated blood vessel, as well as to control the actuators and means of proximal and distal heads for holding in place and anchoring the graft ends. Due to the action of this lever the proximal and distal ends of the graft are simultaneously anchored to the blood vessel wall.

At least one additional tube connects the distal head and control lever. This tube is rigid in longitudinal direction and relatively flexible in lateral direction and has a through axial hole.

At least one flexible control element passes through through axial holes of proximal head and distal head, intermediate head and additional head and operatively connects actuators of proximal head and distal head with the control lever.

The graft delivered and fixated by means of the claimed system may be configured as a tube from biologically compatible material, substantially from foam polytetrafluoroethylene—ePTFE (Teflon). The graft may have an anti-trombogenious, substantially metal coating at least on one its surface, and namely the recipient's bloodstream facing surface. This coating may substantially contain titanium. For delivery within a recipient's blood vessel, the graft is set onto the intermidiate tube of delivery device and secured by its ends to the proximal head and distal head of this device.

The claimed set of assembly units for assembling at least one graft delivery means additionally contains a venous filter secured at the free end of its proximal head and serving for removal of emboli and cholesterol patches from the operated blood vessel.

The proximal head of the claimed device comprises a cyindrical stepped body with a through axial hole of variable section. This body has a central part with an outer diameter corresponding to the graft inner diameter, proximal part and distal part. The proximal part has an outer diameter smaller than the outer diameter of the central part, and at its free end forms a bush with external thread. The distal part has a shank for securing the intermediate tube. The proximal head has means for holding in place the graft proximal end, the means being configured as valleys for fixing graft end disposed on the end face of the body central part, in the region of its transition into proximal part, and a spring loaded fixing bush with ridges on its end face opposite to said valleys. This spring loaded fixing bush is located coaxially with the proximal part of cylindrical stepped body to reciprocate towards the body central part.

The proximal head also has means for securing the graft proximal end to a blood vessel wall, substantially U-shaped staples disposed in radial slots on the end face of the body central part, these slots alternating with said valleys. These means are formed as rotatable levers for radialyy extending said U-shaped staples. The rotatable levers are pivotally secured by one its end in through longitudinal slots evenly arranged in the proximal part of the body, parallel with its axial hole. The rotatable levers are adapted to cooperate via the free end of its outer surface with the middle part of said U-shaped staples, and via its inner surface—with the proximal head actuator.

The proximal head also has a hold down bush with a thread on its inner surface, this bush being set on the threaded bush of the free end of said body proximal part, coaxially with the spring loaded fixing bush. The hold down bush is adapted to reciprocate along the thread to adjust tightening force of spring loaded fixing bush to the end face of the body central part.

An actuator shaped as a cylinder with a through axial stepped hole and conical working surface is mounted in the through axial hole of the cylindrical stepped body of said proximal head to reciprocate in this hole and periodically cooperate by its conical working surface with inner surfaces of the rottable levers for radially extending U-shaped staples. This actuator is rigidly secured on said flexible control element passing through the through axial holes of proximal head, distal head, intermediate tube and additional tube, and operatively connecting actuators of proximal head and distal head with the control lever.

Valleys in the proximal head for fixing the graft proximal end are located substantially on the edge of the end face of said body central part, in the area of its stepped transition to the proximal part of this body.

Each of radial slots comprised in the proximal head for locating fastener means, substantially U-shaped staples, terminates in a means for setting apart these staples formed as two curved ducts disposed in the end face of the central part of aforesaid body symmetrically with its radial axis and sufficiently close together. Ther curved ducts diverge from one another in direction from the through axial hole of the body to its outer surface. Each U-shaped is disposed by its middle part within tye through axial hole of this body, and by its free ends—in two curved ducts so as to extend these free ends in direction from the inside to the outside due to radial force applied to a U-shaped staple from within the body hole. Thereby the staple is withdrawn from the curved ducts, pierces the graft wall and that of a corresponding blood vessel and comes out onto the surface of this blood vessel to bend on this surface and so fix the graft position relative to this graft vessel.

The proximal head also has rotatable levers to radially extend U-shaped staples which are pivotally attached by one its end in through longitudinal slots of the proximal part of cylindrical stepped body. The rotatable levers are adapted to cooperate by the free end of its outer surface with the middle part of U-shaped staples, and by its inner surface—with the proximal head actuator. They have at least two different lengths, whereby the number of levers that may be located, and hence also the number of U-shaped staples extended by these lever, increases.

Thd spring loaded fixing bush is located in the proximal head coaxially with the proximal part of aforesaid cylindrical stepped body to reciprocate towards the body central part. It is provided with ridges on its end face presented to the end face of this body central part. The ridges are opposite to the vballeys and means for setting apart the ends of U-shaped staples and serves to fixate the corresponding graft end U-shaped staples in the course of graft delivery.

The distal head of claimed device comprises a cylindrical stepped body with a through axial hole of variable section. This body has a central part with outer diameter corresponding to the inner diameter, proximal part and distal part. The body distal part has an outer diameter smaller than the outer diameter of this central part, and at its free end forms bush with external thread. The body proximal part is provided with a shank for securing an intermediate tube.

The distal head has means for holding in place the graft distal end, these means formed as valleys for fixation of the graft end. Thus valleys are disposed on the end face of the body central part, in the area of its stepped transition to the distal part, as well as a spring loaded fixing bush with ridges on its end face opposite to aforesed valleys. The spring loaded fixing bush is disposed coaxially with the distal part of said cylindrical stepped body to reciprocate towards the body central part.

The distal head is provided with means for securing the graft distal end to a blood vessel wall via fastener means, substantially U-shaped staples which are located in radial slots on the end face of the body central part, these slots alternating with valleys. These means are rotatable levers for radially extending U-shaped staples. The rotatable levers are pivotally mounted, via one its end in through longitudinal slots evenly arranged in the distal part of aforesaid body, parallel to its through axial hole. The rotatable levers are adapted to cooperate by the free end of its outer surface with the middle part of U-shaped staples, and by the inner surface—with the distal head actuator.

The distal end also has a hold down bush with a thread on its inner surface mounted on a thread bush of the free end of this body distal part, coaxially with the spring loaded fixing bush. The hold down bush is adapted to reciprocate along the thread to adjust the force of tightening this spring loaded fixing bush to the end face of the body central part.

The distal head comprises an actuator shaped as a cylinder with a through axial stepped hole and conical working surface. It is disposed in the through axial hole of this cylindrical stepped body to reciprocate in this hole and periodically cooperate by their conical working surface with inner surfaces of rotatable levers for radially extending U-shaped staples. This actuator is adapted to reciprocate on a flexible control element passing via through axial holes of the proximal head, distal head, intermediate tube and additional tube and operatively connects actuators of proximal head and distal head with the control lever.

In the distal head valleys for fixating a graft distal end are located substantially on the edge of the end face of aforesaid body central part, in the are of its stepped transit to this body distal part.

Each of radial slots of the distal head for locating fastener means, substantially U-shaped staples, terminates in a means for setting apart these staples. It contains two curved ducts disposed in the end face of aforesaid central part of the body symmetrically to its radial axis and sufficiently close together. The curved ducts diverge from one another in direction from from the through axial hole of of this body to its outer surface. Each U-shaped staple is disposed by its middle part within the through axial hole of the body, and by its free ends—in two curved ducts to extend these free ends in direction from the inside to the outside due to radial force applied to this U-shaped staple from within the body hole. Thereby the staple is withdrawn from the curved ducts, pierces the wall of a graft and corresponding blood vessel and comes out onto the outer surface of this blood vessel to bend thereon and thus fixate the graft location relative to this blood vessel.

Rotatable levers of the distal head serving for radially extending U-shaped staples are pivotally mounted by one its end in through longitudinal slots of the distal part of aforesaid cylindrical stepped body. These levers are adapted to cooperate by the free end of its outer surface with the middle part of U-shaped staples, and by its inner surface—with the distal head actuator and have at least two different lengths. Thereby the number of levers that may be located, and hence also the number of U-shaped staples simultaneously extended by tjhese levers.

The distal head also has a spring loaded fixing bush located coaxially with the distal part of the cylindrical stepped body to reciprocate towards the body central part. This bush is provided with ridges on its end face presented to the end face of the body central part. The ridges are opposite to valleys and means for setting apart the ends of U-shaped staples and serve to fixate accordingly the graft end and and U-shaped staples in the process of graft delivery.

The claimed device for graft delivery has at least one control lever used to control the process of delivering grafts within an operated blood vessel as well as to operate the actuators of proximal head and distal head. The control lever contains a hollow body having a proximal end, distal end with a perpendicular slot and a longitudinal axial duct wherefrom within the body, near its distal end there extend, at an acute angle, two additional ducts. All these ducts extend within the perpendicular slot. There is also a bush with a shank at its proximal end and with a through axial hole located at the body proximal end. Within the longitudinal axial duct of the body there is disposed a spring-loaded pusher with a through axial hole and two longitudinal slots which are open in opposite directions. This spring-loaded pusher is disposed so as to reciprocate within the longitudinal axial duct of aforesaid body, provided with a lock and operatively connected via an additional hold-down element with the actuator of said distal head. The control lever also has a curved L-shaped lever pivotally secured by the middle of its short arm in perpendicular slot of said body and provided with a first and second pivot pin at the ends of this short arm. The first pivot pin adjoining the long arm of L-shaped lever is operatively connected with the spring-loaded pusher, whereas the second pivot pin is operatively connected with at least one flexible control lever passing via through axial holes of proximal and distal head, intermediate, additional tube and spring-loaded pusher and operatively connecting the actuators of proximal and distal head with the control lever.

The claimed device has at least one flexible control element passing via through axial holes of proximal, distal head, intermediate, additional tube and spring-loaded pusher and and operatively connecting actuators of proximal and distal head with a control lever. This flexible control lever may be formed substantially as a wire, thin cord or plastic thread.

The additional hold down element operatively connecting the spring loaded pusher of control lever with distal head actuator is connected therewith by its corresponding ends and is shaped as a tube member being rigid in longitudinal direction and relatively flexible in lateral direction, in particular, a plastic tube or flexible hollow shaft having a through axial hole for a flexible control element.

There is also claimed a method for graft delivery and anchoring within a recipient's blood vessel via the claimed system. This method includes several successive steps. First, there are defined individual dimensions of the delivered graft, in particular, the diameter and length of this graft according to recipient's clinical state and angiographic measurements of its operated blood vessel. The there is assembled, on the basis of the set of assembly units, at least one device for delivering a graft of individual dimensions within a recipient's blood vessel and simultaneous anchoring of this graft at both its sides on the blood vessel wall. To do this there are chosen a proximal and distal head of a corresponding diameter, intermediate and additional tube of a corresponding length, as well as a control lever and flexible control element and, and these are connected in such a way that the flexible control element passes via through axial holes of proximal, distal head, intermediate and additional tube and operatively connects the actuators of proximal and distal head with the control lever. The assembled device is provided with a vein filter mounted on the actuator of proximal head on the side of its hold down bush. Then the delivered graft is located outside the intermediate tube and its ends are fixated in proximal and distal head. The graft delivery and anchoring system is ready for work.

The system thus assembled is held by the control lever and inserted within the recipient's blood vessel, simultaneously performing X-ray control of location of this system relative to this blood vessel. The vein filter mounted at the system free end traps the encountered emboli and cholesterol patches. When the system comes into required position, the L-shaped lever of control lever is pressed to cause movement of actuators of proximal and distal head towards one another ejecting in radial direction U-shaped staples of both heads and suturing the graft on both its sides. The U-shaped staples comong out of these heads loosen by their middles the ridges of spring-loaded fixing bushes of proximal and distal head to draw these bushes aside and release graft ends clamped in aforesaid heads.

At the last stage the system is withdrawn from the graft sutured on both its sides to the blood vessel, and also from the blood vessel. Hence the vein filter pressed by its end to the end of hold down bush of the proximal head is locked by this bush, and its contents sealed in this filter. At the last stage there are performed utilization of the removed single-use system and checking the operation results.

### BRIEF DESCRIPTION OF THE DRAWINGS

The invention will now be described in conjunction with the following drawings in which like reference numerals designate like elements and wherein:

FIG. 1 shows a general view of the system for graft delivery and anchoring to a blood vessel wall;

FIG. 2 shows a side view of the system for graft delivery and anchoring to a blood vessel;

FIG. 3-5 show respectively a general view, side view and longitudinal section of the proximal head;

FIGS. 6, 7 show respectively a general view and side view of the proximal head means for anchoring the graft to a blood vessel wall;

FIGS. 8, 11 show respectively a general view, side view, longitudinal section and view of the middle of proximal part near the proximal head;

FIGS. 12, 13 show respectively a general view and side view of the fixing bush;

FIG. 14-16 show respectively a general view and side views of rotatable levers;

FIGS. 17, 18 show the actuators respectively of the proximal and distal head;

FIG. 19-21 sow respectively a general view, side view and longitudinal section of the distal head;

FIGS. 22, 23 show respectively a general view and side view of the distal head means for securing a graft to the blood vessel wall;

FIG. 24-26 show respectively a general view, side view and longitudinal section of the control lever;

FIG. 27 shows a longitudinal section of the spring loaded pusher of the control lever.

### DETAILED DESCRIPTION OF PREFERRED EMBODIMENTS

The preferred embodiments of the present invention are described below. The inventors of the present subject matter contemplate that the embodiments described herein are capable of use in the repair of blood vessels and in other procedures. Thus, it is intended that the present invention cover the modifications and variations of the invention, provided they come within the scope of the appended claims and their equivalents.

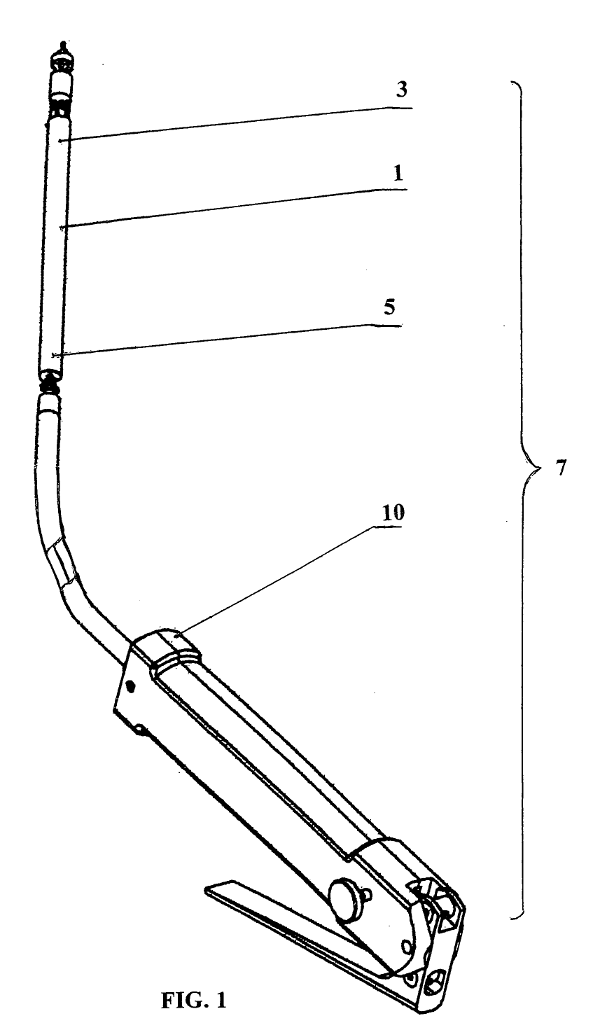

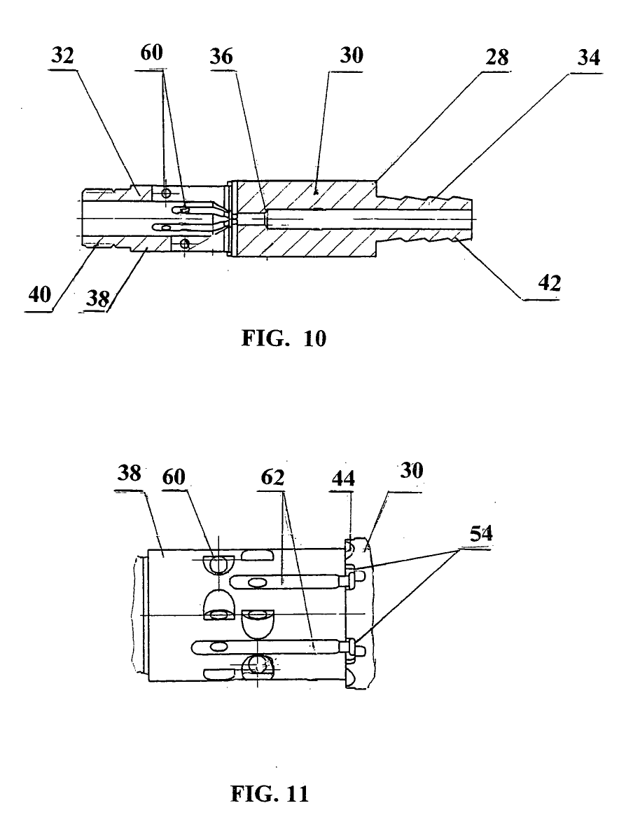

The most preferred embodiments of a delivery system, according to the present invention, are shown in drawing FIGS. 1-27.

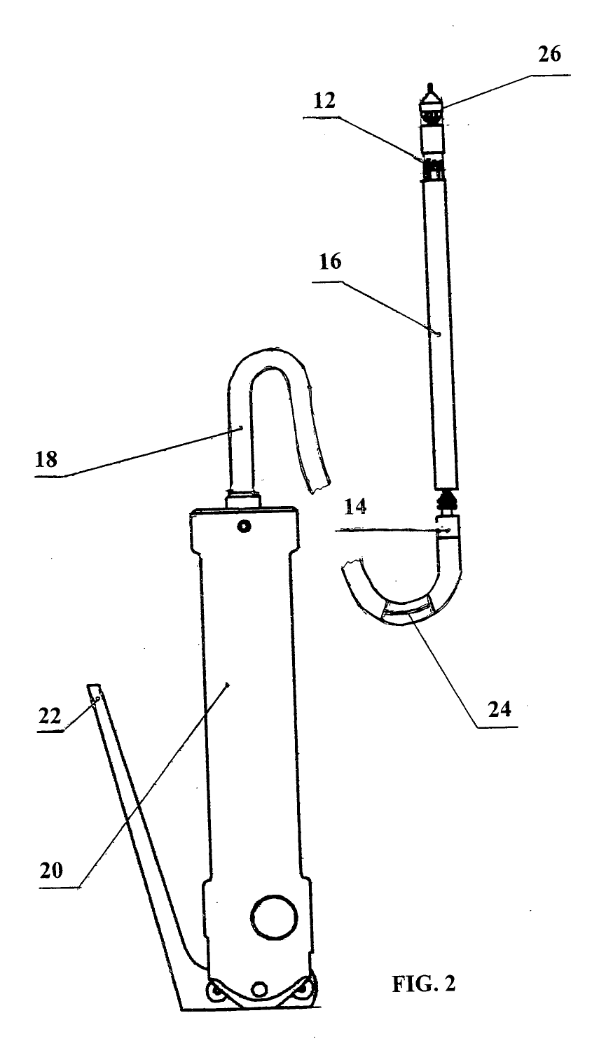

The claimed system for delivering and anhoring a graft of individual dimensions (FIGS. 1, 2) comprises: a) graft 1 shaped as a tube from biologically compatible material and having a proximal 3 and distal 5 end, as well as b) a set of assembly units 7 for its assembly on the basis of at least one device 10 for delivering graft 1 within a recipient's blood vessel and simultaneously securing this graft 1 on both its sides to a blood vessel wall.

Device 10 for delivering graft 1 (FIG. 2) comprises: at least one proximal 12 and at least one distal 14 head which serve for holding graft 1 in the process of its delivery within a blood vessel, as well as for securing the proximal 3 and distal 5 end of graft 1 to the wall of an operated blood vessel. The proximal 12 and distal 14 heads have a through axial hole with an actuator located therein, as well as means for holding a respective end of graft 1 and means for securing this end of graft 1 to a blood vessel wall (in more detail they are described below). Besides, the claimed device 10 for delivering graft 1 assembled on the basis of a set of assembly units 7 contains at least one intermediate tube 16, at least one additional tube 18, at least one control means—control lever 20 with an L-shaped lever 22, as well as at least one flexible control means 24 (FIG. 2). Intermediate 16 and additional 18 tube are rigid in longitudinal direction and relatively flexible in lateral direction and have a through axial hole.

Control lever 20 with an L-shaped lever 22 serves for controlling the process of inserting device 10 for delivering graft 1 within an operated blood vessel, as well as for controlling the actuators and and means of proximal 12 and distal 14 head for holding and securing the ends of graft 1. Flexible control element 24 passes via through axial holes of proximal 12, distal head 14, intermediate 16 and additional 18 tube and operatively connects actuators of proximal 12 and distal 14 head with control lever 20. Due to the action on control lever 20 with L-shaped lever 22 proximal 3 and distal 5 end of graft 1 are simultaneously secured to the blood vessel wall.

Device 10 for delivering graft 1 (FIG. 2) additionally comprises a vein filter 26 mounted at the free end of its proximal head 12 and serving for removing emboli and cholesterol patches from the operated blood vessel.

Graft 1 delivered and fixated via claimed system 10 may be shaped as a tube from biologically compatible material, substantially foam polytetrafluoroethylene—ePTFE (tephlon). Graft 1 may have an antithrombogenic, substantially metal coating at least on one its bloodstream facing surface. This coating may be substantially from titanium. For delivery within a recipient's blood vessel, graft 1 is set on an intermidiate tube 16 of delivery device 10 and secured by its ends on proximal 12 and distal 14 head of this device 10.

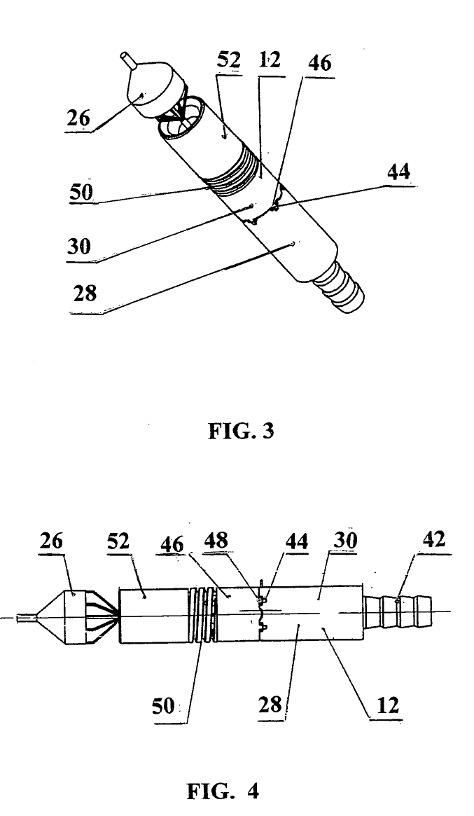

FIG. 3-5 show proximal head 12 of a graft delivery device with a vein filter 26 at its free end. The main part of proximal head 12 of the claimed device 10 (FIG. 3-11) is a cylindrical stepped body 28. This body 28 (FIG. 10) has a central part 30 with an outer diameter corresponding to the inner diameter of graft 1, proximal 32 and distal 34 part, as well as a through axial hole 36 of variable section. Proximal part 32 (FIG. 10) has an outer diameter smaller than the outer diameter of the central part 30, and at its free end forms a bush 38 with external thread 40. Distal part 34 (FIG. 10) is provided with a shank 42 for securing intermediate tube 16. Proximal head 12 (FIG. 3-5) also has means for holding the proximal end of graft 1 containing valleys 44 for fixating the end of graft 1, the valleys being disposed on the end face of central part 30 of this body, at the region of its transition to proximal part 32, as well as a spring-loaded fixing bush 46 with ridges 48 on its end face opposite to said valleys 44 (FIGS. 3, 4 and 12, 13). This fixing bush 46 loade by spring 50 is disposed coaxially with proximal part 32 of cylindrical stepped body 28 to reciprocate towards its central part 30.

Besides spring loaded fixing bush 46 (FIGS. 3, 4, 5), proximal head 12 also has a hold-down bush 52 with a thread on its inner surface wich is mounted on bush 38 of the free end of proximal part 32 of body 30 coaxially with spring loaded fixing bush 46. Hold-down bush 52 is adapted to reciprocate along thread 40 to adjust the force of tightening spring loaded fixing bush 46 to the end face of central part 30 of body 28.

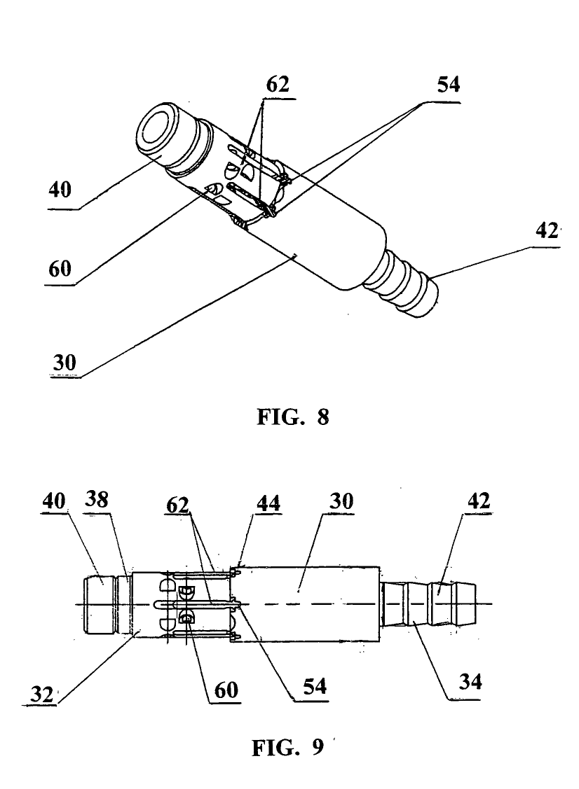

Proximal head 12 has means for securing the proximal end of graft 1 to the blood vessel wall via fastener means, substantially U-shaped staples 100 located in radial slots 54 (FIGS. 8, 9) on the end face of central part 30 of the body alternating with said valleys 44. These means are configured as rotatable levers 56 for radially extending said U-shaped staples 100. Rotatable levers 56 (FIGS. 6, 7) are pivotally secured by one its end on pivot pins 58 in holes 60 made in the walls of through longitudinal slots 62 (FIGS. 8, 9 and 11) evenly arranged in proximal part 48 of the body, parallel with its through axial hole 36. Rotatable levers 56 (FIGS. 6, 7 and 14-16) are adapted to cooperate by free end 64 of its outer surface with the middle part of said C-shaped staples 100, and by its inner surface 66—with the actuator 68 of proximal head 12.

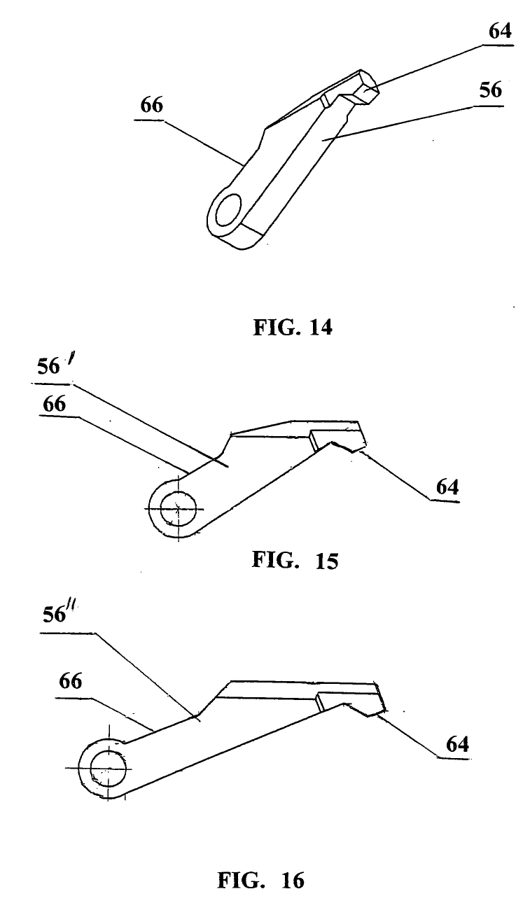

Rotatable levers 56 (FIG. 14-16) have at least two different lengths—short levers 56′ and long levers 56″, whereby there is increased the number of located levers, and hence, also the number of simultaneously the number of U-shaped staples extended by these levers.

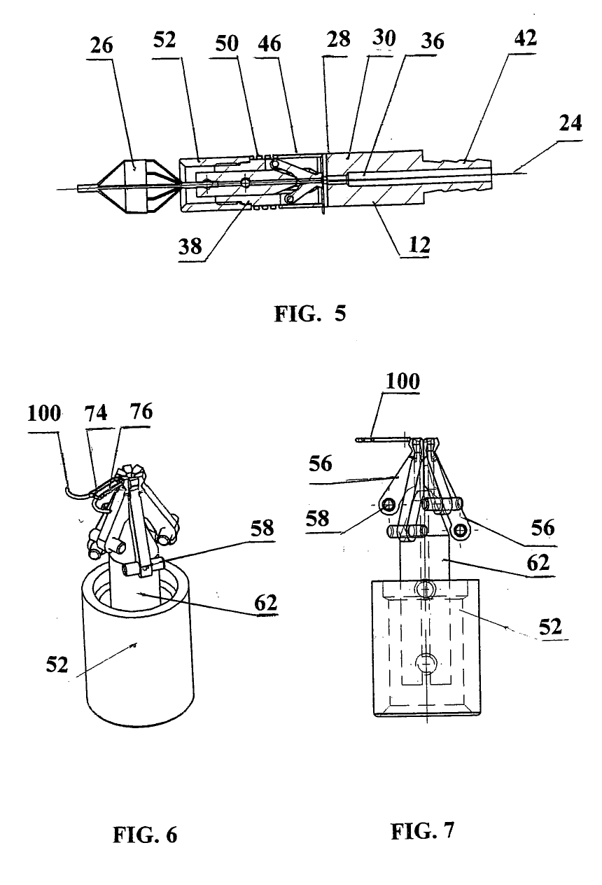

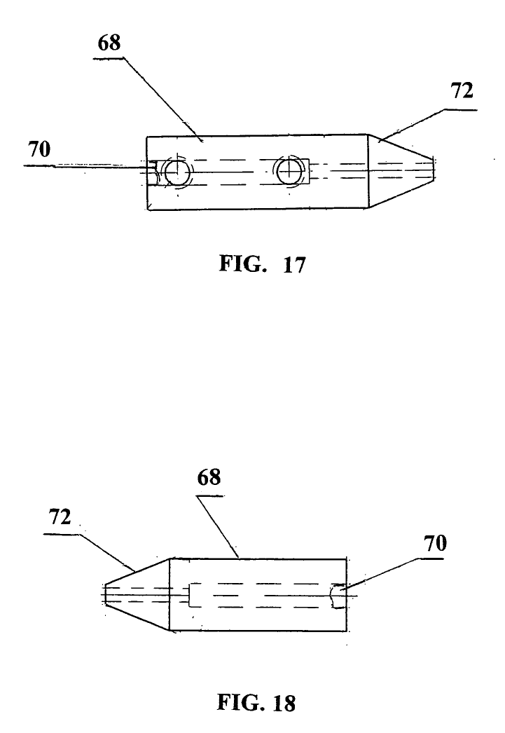

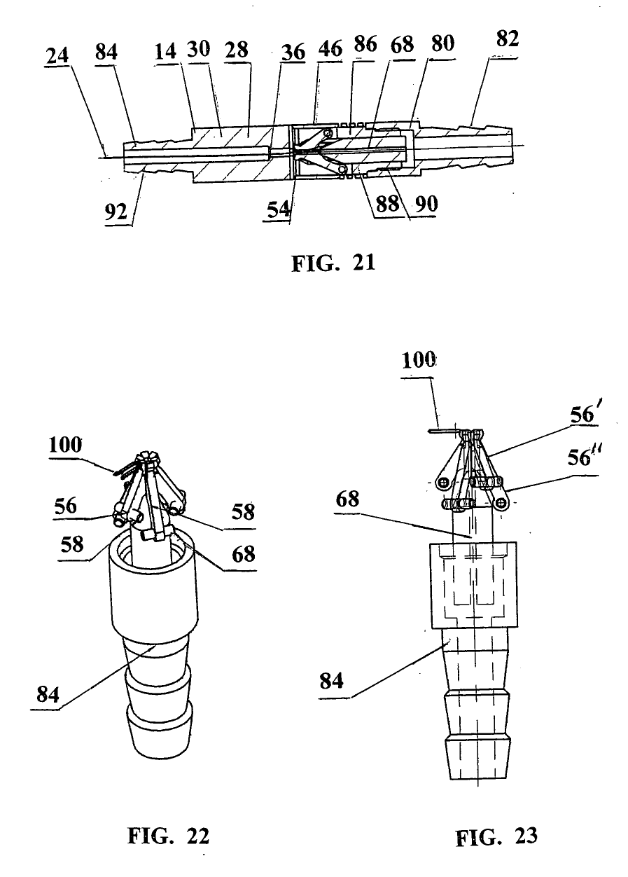

Actuators 68 (FIGS. 17, 18) shaped as a cylinder with a through axial stepped hole 70 and conical working surface 72 are located in through axial holes of cylindrical stepped bodies of proximal head 12 and distal head 14 to reciprocate in these holes and periodically cooperate by its conical working surface 72 with inner surfaces 66 of rotatable levers 56 for radially extending U-shaped staples 100. These actuators 68 are located on a flexible control element 24 passing via through axial holes of proximal 12, distal 14 head (FIGS. 5, 21), intermediate 16 and additional tube 18 and operatively connecting actuators 68 of proximal 12 and distal 14 head with control lever 20.

Each of radial slots 54 of proximal head 12 for locating fastener means, substantially U-shaped staples 100, terminate in a means for setting apart the ends of these staples configured as two curved ducts 74 and 76 (FIG. 6) located in the end face of central part 30 of body 28 symmetrically to the radial axis of said body and sufficiently close together. Curved ducts 74 and 76 are diverging from one another in direction from the through axial hole 36 of body 28 to its outer surface. Each U-shaped staple 100 is located by its middle part within the through axial hole 36 of body 28, and by its free ends—in two curved ducts 74 and 76 to extend these free ducts in direction from the inside to the outside due to axial force applied to U-shaped 100 from within the hole of of body 28. Thereby U-shaped staple 100 is withdrawn from curved ducts 74 and 76 and pierces the wall of graft 1 and of a corresponding blood vessel and comes out onto the outer surface of this blood vessel to bend thereon and thus fixing the location of graft 1 relative to this blood vessel.

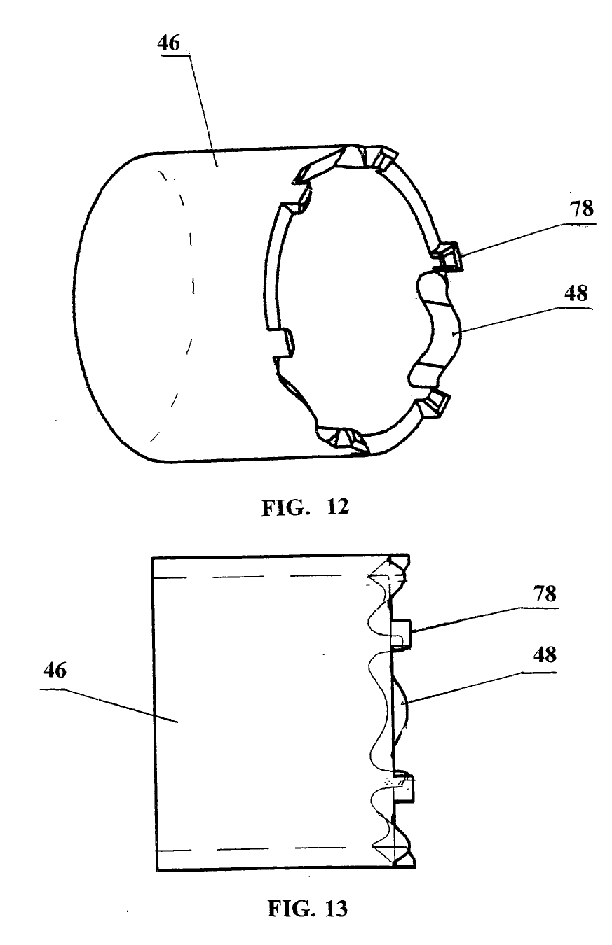

Spring loaded fixing bush 46 (FIGS. 12, 13) has ridges 48 and 78 on its end face presented to the end face of central part 30 of body 28. Ridges 48 are opposite to valleys 44 and serve to fixate the proximal end of graft 1. Ridges 78 are opposite to means for setting apart the ends of U-shaped staples 100—curved ducts 74 and 76 and serve to fixate U-shaped staples 100 so that they should not fall out in the process of delivering graft 1.

The distal head 14 of the claimed device, except for hold-down bush 80, is completely identical to proximal head 12, it is turned around relative to it at 180 degrees to the opposite side (FIGS. 19, 20, 21). Hold-down bush 80 of distal head 14 differs from hold-down bush 52 of proximal head 12 only in that it has at its free end a shank 82 for attachment to additional tube 19 (FIGS. 19, 20, 21). Distal head 14 (FIGS. 19, 20, 21) also contains a cylindrical stepped body 28 with a through axial hole 36 of variable section. This body has a central part 30 with an outer diameter corresponding to the inner diameter of the graft, proximal 84 and distal 86 part (FIG. 21). Distal part 86 of the body has an outer diameter smaller than the outer diameter of central part 30, and at its free end forms a bush 88 with external thread 90. Proximal part 84 of the body is provided with a shank 92 for securing an intermediate tube.

Distal head 14 (FIGS. 19, 20, 21) is provided with means for holding the distal end of graft 1 shaped as valleys 44 for fixating the end of graft 1 and located on the end face of central part 30 of body 28, in the region of its stepped transition to distal part 86, as well as a spring-loaded fixing bush 46 with ridges 48 on their end face opposite to these valleys 44. Spring-loaded bush 46 is disposed coaxially to distal part 86 of cylindrical stepped body 28 to reciprocate towards central part 30 of body 28.

Distal head 14 (FIGS. 22, 23) has means for securing distal end 5 of graft 1 to a blood vessel wall via fastener means, substantially U-shaped staples 100 which are located in radial slots 54 on the end face of central part 30 of body 28 alternating with valleys 44. These means are configured as rotatble levers 56 for raially extending U-shaped staples 100. Rotatable levers 56 are pivotally mounted by one its end in through longitudinal slots 62 evenly arranged in distal part 86 of body 28, parallel with its through axial hole 36. Rotatable levers 56 are adapted to cooperate by the free end 64 of its outer surface with the middle part of U-shaped staples 100, and by its inner surface 66—with actuator 68 of distal head 14.

Distal head 14 (FIG. 21) also has a hold-down bush 80 with a thread on its inner surface set on thread 90 of bush 88 of the free end of distal part 86 of body 28, coaxially with spring-loaded bush 46. Hold-down bush 80 is adapted to reciprocate over thread 90 to adjust the force of tightening the spring-loaded fixing bush 46 to the end face of central part 30 of body 28.

Distal head 14 contains an actuator 68 (FIG. 21) formed as a cylinder with a through axial stepped hole 70 and conical working surface 72. It is located in a through axial hole 36 of cylindrical stepped body 28 to reciprocate in this hole and periodically cooperate by its conical working surface 72 with inner surfaces 66 of rotatable levers 56 for radially extending U-shaped staples. This actuator 68 is adapted to reciprocate on the flexible control element 24 passing via through axial holes 36 of proximal 12 and distal 14 head, intermediate 16 and additional 18 tube and operatively connecting actuators 68 of proximal 12 and distal 14 head with control lever 20.

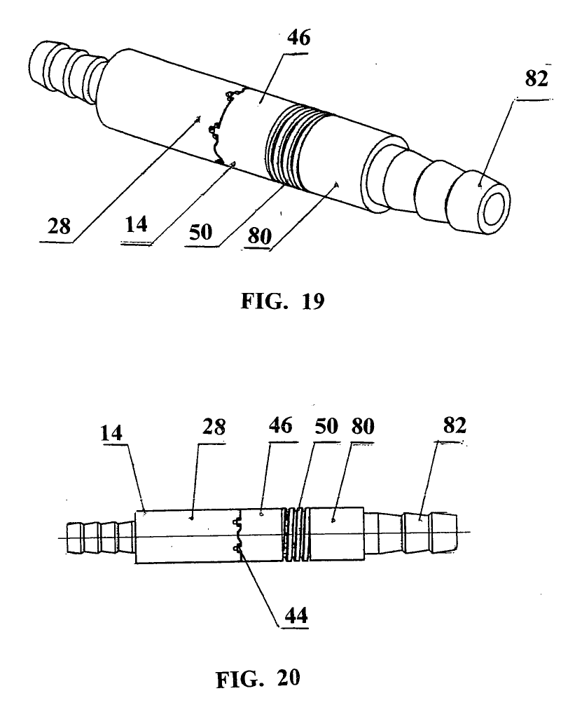

In distal head 14 valleys 44 for fixating the distal end 5 of graft 1 are located substantially on the edge of end face of central part 30 of body 28, in the region of its stepped transition to distal part 86 (FIGS. 20, 21).

Each of radial slots 54 of distal head 14 adapted to receive fastener means, substantially U-shaped staples 100 terminates in a means for setting apart the ends of these staples (FIGS. 22, 23). It is formed as two curved ducts 74, 76 located in the end face of central part 30 of body 28 symmetrically with radial axis of the latter and sufficiently close to one another. Curved ducts 74, 76 diverge from one another in direction from through axial hole 36 of body 28 to its outer surface. Each U-shaped staple 100 is disposed by its middle part within through axial hole 36 of the body, and by its free ends—in two curved ducts 74, 76 so as these free ends may extend in direction from the inside to the outside due to radial force applied to U-shaped staple 100 from within hole 36 of this body. Thereby staple 100 is withdrawn from curved ducts 74, 76, pierces the wall of graft 1 and of a corresponding blood vessel and comes out onto the outer surface of this blood vessel to bend thereon and thus fix graft 1 relative to this blood vessel.

Rotatable levers 56 of distal head 14 which serve to radially extend U-shaped staples 100 are pivotally secured by one its end in through longitudinal slots 62 of distal part 86 of cylindrical stepped body 28 (FIGS. 20, 21). These levers 56 are adapted to cooperate by free end 64 of their outer surface with with the middle part of U-shaped staples 100, and by their inner surface 66 with actuator 68 of distal head 14 and have at least two different lengths. Threby the number of levers 56 that may be located, and hence the number of U-shaped staples 100 simultaneously extended by these levers increases.

Distal head 14 also has a spring-loaded fixing bush 46 (FIGS. 19, 20, 21) completely identical with the fixing bush of proximal head 12 and disposed coaxially with distal part 86 of cylindrical stepped body 28 to reciprocate towards central part 30 of body 28. This bush 46 has ridges 48 and 78 on its end face presented to the end face of central part 30 of body 28. Exactly as in proximal head 12, ridges 48 are opposite to valleys 44, and ridges 78—to means for setting apart the ends of U-shaped staples 100—ducts 74 and 76 and serve to fixate respectively the end of graft and U-shaped staples 100 in the process of delivering graft 1.

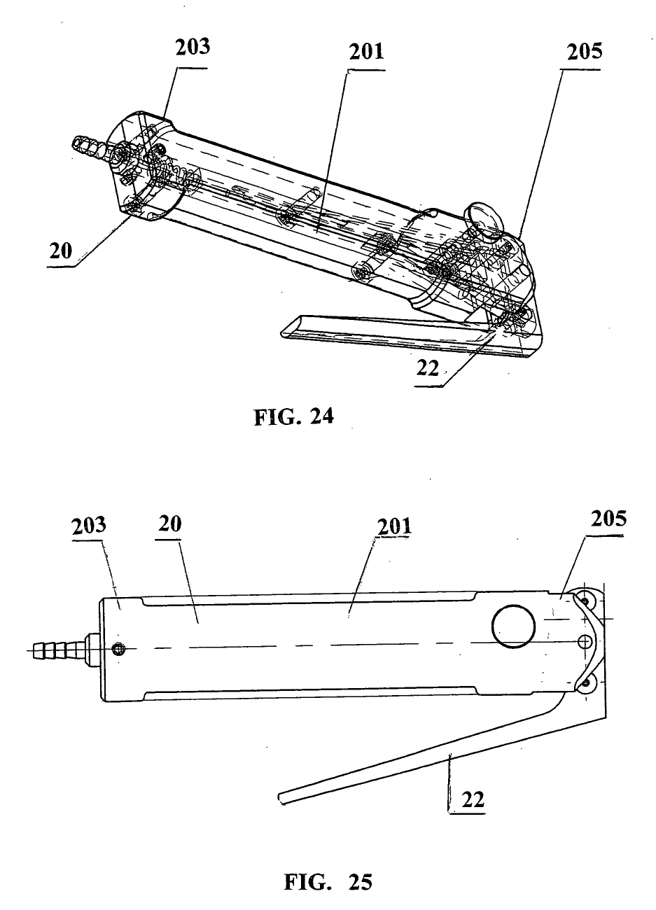

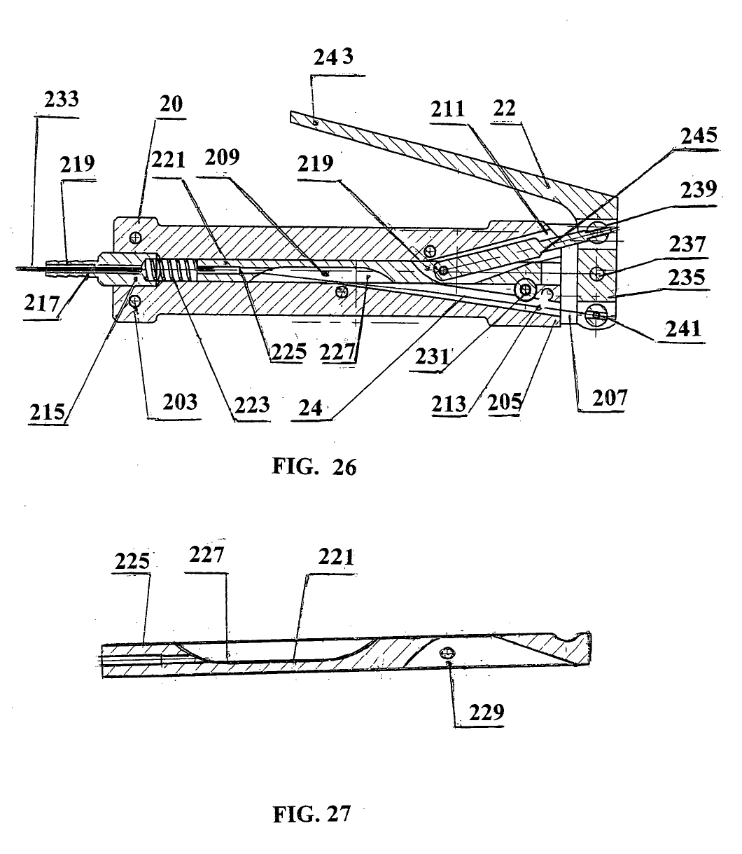

The claimed device 10 for delivering graft 1 has at least one control lever 20 for controlling the process of inserting the device for delivering grafts 1 within an operated blood vessel, as well as for controlling the actuators of proximal 12 and distal 14 head. Control lever 20 (FIG. 24-27) contains a hollow body 201 having a proximal end 203, distal end 205 with a perpendicular slot 207 and a longitudinal axial duct 209 wherefrom, within body 201, near its distal end 205, there extend at an acute angle two additional ducts, 211 and 213 (FIG. 26). All these ducts 209, 211 and 213 extend within perpendicular slot 207. There is also a bush 215 with shank 217 at its proximal end and a through axial hole 219 disposed at the proximal end 203 of body 201. Within the longitudinal axial duct 209 of body 201 there is reciprocably disposed a spring-loaded pusher 211 with spring 223, through axial hole 225 and two longitudinal slots, 227 and 229 open on opposite sides (FIG. 27). This spring-loaded pusher 211 is provided with a lock 231 and operatively connected via an additional hold-down element 233 with actuator 68 of distal head 14. The additional hold-down element 233 is connected with spring-loaded pusher 221 and actuator 68 of distal head 14 and shaped as a tube member, rigid in longitudinal direction and relatively flexible in lateral direction, such as a plastic tube or flexible hollow shaft having a through axial hole for the flexible control element 24. Control lever also has a bent L-shaped lever 22 pivotally mounted by the middle of its short arm 235 on a pin 237 in perpendicular slot 207 of the body and provided with a first and second pivot pin, respectively 239 and 241 at the ends of this short arm 235. The first pivot pin 239 adjoining the long arm 243 of this L-shaped lever is operatively connected via lever 245 with spring-loaded pusher 221, whereas the second pivot pin 241 is operatively connected at least with one flexible control element 24 passing via through axial holes of proximal 12, distal 14 head, intermediate 16, additional 18 tube, additional hold-down element 233 and spring-loaded pusher 221 and operatively connecting actuators 68 of proximal and distal heads 12 and 14 with control lever 20. The claimed device has at least one flexible control element 24 which may be formed substantially as a wire, thin cord or plastic thread.

The claimed system for graft delivery and anchoring operates on the basis of the claimed method as follows. First there is assembled device 10 for delivering graft 1, and to his end there are defined individual dimensions of delivered graft 1, such as its diameter and length, considering the clinical state of a recipient and angiographic measurements of its operated blood vessel. Then, there is performed the assembly, on the basis of the set of assembly units 7, of at least one device 10 for delivering graft 1 of individual dimensions within the recipient's blood vessel wall and simultaneously anchoring this graft 1 at both its ends to the blood vessel wall. To do so, there are selected a proximal 12 and distal 14 head of a corresponding diameter, intermediate 16 and additional 18 tube of a corresponding length, as well as control lever 20, additional hold-down element 233 and flexible control element 24 and connects them together in such a way, that flexible control element 24 passes via through axial holes of proximal 12, distal 14 head, intermediate 16, additional tube 18, additional hold-down element 233 and spring-loaded pusher 221 and operatively connects actuators 68 of proximal 12 and distal 14 head with control lever 20. The assembled device 10 is provided with a vein filter 26 secured on actuator 68 of proximal head 12 on the side of its hold-down bush 52. Then the delivered graft 1 is set outside the intermediate tube 16 and its ends are fixated in proximal 12 and distal 14 head. The system for delivering and anchoring graft 1 is ready for operation.

The system thus assembled is inserted, being held by control lever 20, within the recipient's blood vessel wall, simultaneously performing X-ray control of this system location relative to this blood vessel. Vein filter 26 mounted at the system free end traps emboli and cholesterol patches met with on its way.

Once the system has reached the desired position in the blood vessel, L-shaped lever 22 of control lever 20 is pressed. In this case lever 22 turns in slot 207 about pin 237, wehereby the pin 239 of lever 22 approaches the end of lever 20, and pin 241 moves away from this lever end. This movement of lever 22 is transmitted via pin 239 and lever 245 to spring-loaded pusher 221 and additional hold-down element 233 connected with the pusher, and via pin 241—to the flexible control element 24, whereby actuators 68 of proximal 12 and distal 14 head move towards one another to eject in radial direction U-shaped staples 100 of both heads and suture graft 1 at both its ends. The ejecting U-shaped staples 100 press by their middle on the ridges of spring-loaded fixing bushes 46 or proximal 12 and distal 14 head to move these bushed 46 aside and release the ends of graft 1 clamped in said heads.

At the final stage the system is withdrawn from graft 1 sutured at both its ends to a blood vessel, as well as from this blood vessel. In this case vein filter 26 pressed by its end to the end of hold-down bush 52 of proximal head 12 is locked by this bush, and its contents—sealed in this filter 26. At the last stage the withdrawn single use system is utilized, and the operation results checked.

The application of claimed system and method allow to provide a medical system solving simultaneously two problems: graft delivery within a blood vessel using thechniques of minimally invasive surgery, as well as secure fixation of a delivered and located graft to the blood vessel walls to prevent its displacement from a given position within a recipient's blood vessel due to peristelsic oscillations of the walls of this blood vessel and oscillations of the recipient's body during his movement.

While this invention has been described in conjunction with specific embodiment thereof, it is evident that many alternatives, modifications and variations will be apparent to those skilled in the art. Accordingly, the preferred embodiments of the invention, as set forth herein are intended to be illustrative, not limiting. Various changes may be made without departing from the spirit and scope of the invention as defined in the following claims.

1. A system for graft delivery and anchoring within a recipient's blood vessel, comprising:
a) a graft shaped as a tube from biologically compatible material, its dimensions, in particular, the diameter and length, being defined by recipient's clinical state and results of angiographic measurements of its operated blood vessel, this graft of individual dimensions having a proximal end and a distal end;
b) a set of assembly units for assemmbling on its base at least one device for delivering a graft of individual dimensions within a blood vessel of a concrete recipient and for simultaneously anchoring this graft at both its ends to the blood vessel wall, this set containing:
i) at least one proximal head for holding a graft in the process of delivery within a blood vessel and for anchoring its proximal end to the wall of operated blood vessel, the proximal head having a through axial hole with an actuator therein, as well as means for holding the graft proximal end and means for anchoring this graft end to the blood vessel wall;
ii) at least one distal head for holding the graft in the process of its delivery within the blood vessel and for anchoring its distal end to the wall of operated blood vessel, the distal head having a through axial hole with an actuator therein, as well as means for holding the graft distal end and means for anchoring this graft end to the blood vessel wall;
iii) at least one intermediate tube connecting said proximal and distal heads, this tube having rigidity in longitudinal direction and relative flexibility in lateral direction and having a through axial hole;
iv) at least one control means configured substantially as a control lever serving for controlling the process of inserting the graft delivery device within an operated blood vessel, as well as for controlling actuators and means of proximal and distal heads for holding and for anchoring the graft ends, whereby said proximal and distal graft ends are simultaneously secured to the wall of this blood vessel;
v) at least one intermediate tube connecting said distal head and control lever, this tube having rigidity in longitudinal direction and relative flexibility and having a through axial hole;
vi) at least one flexible control element passing via said through axial holes of proximal, distal head, intermediate and additional tube and operatively connecting said actuators of proximal and distal heads with the control lever,
whereby there is provided the delivery of a graft of individual dimensions in a required area of the blood vessel and fixation of both ends of this graft to the wall of said blood vessel.

2. A system according to claim 1, wherein said graft is formed as a tube from biologically compatible material, substantially from foam polytetrafluoroethylene—ePTFE (Tephlon).

3. A system according to claim 2, wherein said graft is formed as a tube from biologically compatible material, substsntially from foam polytetrafluoroethylene—ePTFE (Tephlon) with an anti-trombogeneous, substantially metal coating at least on one its bloodstream facing surface.

4. A system according to claim 3, wherein said graft is formed as a tube from biologically compatible material, substantially from foam polytetrafluoroethylene—ePTFE, (Tephlon) with an anti-thrombogeneous, substantially titanium coating, at least on one, bloodstream facing surface.

5. A system according to claim 1, wherein said graft of individual dimensions shaped as a tube from biologically compatible material is set on this intermediate tube of said graft delivery device and secured by its ends on said proximal and distal heads of this graft delivery device.

6. A system according to claim 1, wherein said set of assembly units for assembling on its base at least one device for delivering a graft of individual dimensions within a recipient's blood vessel and simultaneously anchoring this graft at both its ends to the blood vessel wall additionally contains:
vii) a vein filter mounted at the free end of said proximal head of the graft delivery device and serving for cleaning a blood vessel from emboli and cholesterol patches.

7. A system according to claim 1, wherein said proximal head contains:
a) a cylindrical stepped body with a through axial hole of variable section, this body having a central part with an outer diameter corresponding to the inner graft diameter, a proximal and distal part, said proximal part having an outer diameter smaller than the outer diameter of central part, and at its free end forms a bush with external thread, said distal part having a shank for securing said intermediate tube;
b) means for holding the graft proximal end shaped as valleys for fixing the graft end, said valleys being located on the end face of central part of said body, in the region of its stepped transition to proximal part, as well as a spring-loaded fixing bush with ridges on its end face opposite to said valleys, and this spring-loaded fixing bush is disposed coaxially with the proximal part of said cylindrical stepped body to reciprocate towards the central part of this body;
c) means for securing a graft proximal end to a blood vessel wall via fastener means, substantially U-shaped staples located in radial slots on the end face of central part of said body, alternating with said valleys, these means configured as rotatable levers for radially extending said U-shaped staples, these levers being pivotally mounted by one their end in through longitudinal slots evenly arranged in said body proximal part, parallel to its through axial hole, and the rotatable levers are adapted to cooperate by the free end of their outer surface with the middle part of said U-shaped staples, and by its inner surface—with said actuator of the proimal head;
d) a hold-down bush with a thread on its inner surface mounted on a thread bush of the free end of said body proximal part, coaxially with said spring-loaded fixing bush, the hold-down bush being reciprocable over the thread to adjust the force of tightening said spring-loaded fixing bush to said end face of the body central part;
e) an actuator configured as a cylinder with a through axial stepped hole and conical working surface mounted in the through axial hole of said cylindrical stepped body to reciprocate in this hole and periodically cooperate by its conical working surface with the inner surfaces of this rotatable levers for radially extending said U-shaped staples, this actuator being rigidly secured on said flexible control elements passing via said through axial holes of proximal, distal head, intermediate and additional tubes and operatively connecting said actuators of proximal and distal head with said control lever.

8. A system according to claim 7, wherein said valleys for fixating the graft proximal end are substantially disposed on the edge of end face of said central part of the body, in the region of its stepped transition to the proximal part of this body.

9. A system according to claim 7, wherein each of said radial slots for locating fastener means, substantially U-shaped staples, terminates in a means for setting apart the ends of these staples configured as two curved ducts disposed in said end face of the central part of said body symmetrically with its radial axis and sufficiently close to one another, the curved ducts diverging from one another in direction from the through axial hole of said body to its outer surface, each U-shaped staple being disposed by its middle part within said through axial hole of the body, and by its free ends—in two said curved ducts, to extend these free ends in direction from the inside to the outside due to radial force applied to a U-shaped staple from within the body hole, whereby the staple is withdrawn from said curved ducts, pierces the wall of the graft and of a corresponding blood vessel and comes out onto the outer surface of this blood vessel to bend thereon and thus fix the graft location relative to this blood vessel.

10. A system according to claims 7 and 9, wherein rotatable levers for radially extending said U-shaped staples pivotally secured by one its end in through longitudinal slots of the proximal part of said cylindrical stepped body and adapted to cooperate by the free end of its outer surface with the middle part of said U-shaped staples, and by its inner surface—with said actuator of the proximal part, have at least two different lengths, whereby the number of levers that maybe located, and hence the number of U-shaped staples extended by these levers increases.

11. A system according to claim 7, wherein said spring-loaded fixing bush located coaxially with the proximal part of said cylindrical stepped body to reciprocate towards the central part of this body is provided with ridges on its end face presented to the end face of this central part of the body, said ridges being opposite to said valleys and said means for setting apart the ends of said U-shaped staples and serve for fixing respectively the end of graft and U-shaped staples in the process of graft delivery.

12. A system according to claim 2, wherein said distal head contains:
a) a cylindrical stepped body with a through axial hole of variable section, this body having a central part with an outer diameter corresponding to the inner diameter of the graft, a proximal and distal part, said distal part having an outer diameter smaller than the outer diameter of the central part, and at its free end forms a bush with external thread, said proximal part having a shank for securing said intermediate tube;
b) means for holding the graft distal end, said means containing valleys for fixing the graft end located on the end face of said body central part, in the region of its stepped transition to the distal part, as well as a spring-loaded fixing bush with ridges on its end face opposite to said valleys, this spring-loaded fixing bush being disposed coaxially with the distal part of said cylindrical stepped body, to reciprocate in direction to the body central part;
c) means for securing the graft distal end to a blood vessel wall via fastener means, substantially U-shaped staples located in radial slots on the end face of said body central part, alternating with said valleys, these means containing rotatable levers for radially extending U-shaped staples, these levers being pivotally secured by one its end in through longitudinal slots evenly arranged in the distal part of the body, parallel with its axial hole, the rotatable levers adapted to cooperate by the free end of its outer surface with the middle part of said U-shaped staples, and by its inner surface with said distal head actuator;
d) a hold-down bush with a thread on its inner surface set on a thread bush of the free end of the distal part of this body, coaxially with said spring-loaded fixing bush, the hold-down bush adapted to reciprocate along the thread to adjust the force of tightening said spring-loaded fixing bush to said end face of said body central part;
e) an actuator configured as a cylinder with a through axial stepped hole and conical working surface, located in the through axial hole of said cylindrical stepped body to reciprocate in this hole and periodically cooperate by its conical working surface with inner surfaces of said rotatable levers for radially extending said U-shaped staples, this actuator adapted to reciprocate on said flexible control element passing via said through axial holes of proximal, distal head, intermediate and additional tube and operatively connecting said actuators of proximal and distal head with the cotrol lever.

13. A system according to claim 12, wherein said valleys for fixing the graft distal end are located substantially on the edge of said end face of central part, in the region of its stepped transition to the distal part of this body.

14. A system according to claim 12, wherein each of said radial slots for locating fastener means, substantially U-shaped staples, terminates in a means for setting apart the ends of these staples, said means containing two curved ducts disposed in said end face of the central part of said body symmetrically with its radial axis and sufficiently close to one another, the curved ducts diverging from one another in direction from the through axial hole of said body, each U-shaped staple is disposed by its middle part within said through axial hole, and by its free ends—in two said curved ducts to extend these free ends in direction from the inside to the outside due to radial force applied to the U-shaped staple from within the body hole,
whereby the staple is ejected from said curved ducts, pierce the wall of the graft and a corresponding blood vessel, and comes out onto the outer surface to bend thereon and thus fix the graft location relative to this blood vessel.

15. A system according to claim 12, wherein rotatable levers for radially extending said U-shaped staples pivotally secured by one its end in through longitudinal slots of the distal part of said cylindrical stepped body and adapted to cooperate by the free end of its outer surface with the middle part of said U-shaped staples, and by its inner surface—with said actuator of the distal head, have at least two lengths,
whereby the number of levers that may be located, and hence, the number of U-shaped staples simultaneously extended by these levers is increases.

16. A system according to claims 12 and 14, wherein said spring-loaded fixing bush disposed coaxially with the distal part of said cylindrical stepped body to reciprocate in direction to the central part of said body is provided with ridges on its end face presented to the end face of this central part of said body, said ridges being opposite to said valleys and said means for setting apart the ends of said U-shaped staples and serve to fix respectively the end of graft and U-shaped staples in the process of graft delivery.

17. A system according to claim 1, wherein said at least one control lever serving to control thr process of inserting a graft delivery device within an operated vessel, as well as to control the actuators of said proximal and distal head comprises:
a) a hollow body having a proximal end, distal end with a perpendicular slot and a longitudinal axial duct, wherefrom within the body, near its distal end there extend at an acute angle two additional ducts, all said ducts extending within said perpendicular slot;
b) a bush with a shank at its proximal end and with a through axial hole disposed at the proximal end of this body;
c) a spring-loaded pusher with a through axial hole and two longitudinal slots open to opposite sides, this spring-loaded pusher adapted to reciprocate within the longitudinal axial duct of the body, provided with a lock and operatively connected via an additional hold-down element with said actuator of the distal head;
d) a curved L-shaped lever pivotally secured by the middle of its short arm in said perpendicular slot of the body and provided with a first and second pivot pin at the ends of this short arm, the first pivot pin adjoining the long arm of said L-shaped lever being operatively connected with said spring-loaded pusher, whereas the second pivot pin is operatively connected with said at least one flexible control element passing via said through axial holes of the proximal distal head, intermediate, additional tube and spring-loaded pusher and operatively connecting said actuators of the proximal and distal head with control lever.

18. A system according to claim 1, wherein said at least one flexible control element passing via said through axial holes of the proximal, distal head, intermediate, additional tube and and spring-loaded pusher and operatively connecting said actuators of proximal and distal head with control lever is configured substantially as a wire.

19. A system according to claim 1, wherein said at least one flexible control element passing via said through axial holes of proximal, distal head, intermediate, additional tube and spring-loaded pusher and operatively connecting said actuators of the proximal and distal head with the control lever is configured as a thin cord.

20. A system according to claim 1 wherein said at least one control element passing via said through axial holes of the proximal, distal head, intermediate, additional tube and spring-loaded pusher and operatively connecting said actuators of the proximal and distal head with the control lever is shaped substantially as a plastic thread.

21. A system according to claim 17, wherein said additional hold-down element operatively connecting said spring-loaded pusher of the control lever with said actuator of the distal head is connected therewith by its corresponding ends and is shaped as a tube member, said tube member being rigid in longitudinal direction and relatively flexible in lateral direction, such as a plastic tube or flexible hollow shaft having a through axial hole for said flexible control element.

22. A method for graft delivery and anchoring within a recipient's blood vessel via the claimed system comprising the following successive steps:
a) defining individual dimensions of the delivered graft, such as the diameter an length of this graft according to clinical state of a recipient and results of angiographic dimensions of its operated blood vessel;
b) assembling on the basis of said set of assembly units at least one device for dilivering a graft of individual dimensions within the recipient's blood vessel and simultaneously securing this graft on both its ends to the blood vessel wall, and to this end there are selected a proximal head and a distal head of a corresponding diameter, intermediate and additional tube of corresponding length, as well as a control lever and flexible control element and they are connected together in such a way that said flexible control element should pass via through axial holes of proximal, distal head, intermediate and additional tube and operatively connect said actuators of the proximal and distal head with said control lever;
c) providing the assembled device with a vein filter attached to the actuator of said proximal head on the side of its hold-down bush;
d) setting the delivered graft outside the intermediate tube and fixing its ends in said proximal and distal head;
e) inserting the system thus assembled, held by the control lever, within the recipient's blood vessel and X-ray checking of the location of this system relative to the given blood vessel, the vein filter mounted at the free end of said system trapping emboli and cholesterol patches encountered on its way;
f) pressing, when the system has reached the necessary position, said L-shaped lever of the control lever, whereby the actuators of said proximal and distal head move towards one another to eject in radial direction said U-shaped staples of both heads and suture the graft at both its sides, and the ejected U-shaped staples loosen by their middles the ridges of said spring-loaded fixing bushes of said proximal and distal head, moving these bushes aside and releasing graft ends clamped in said heads;
g) withdrawing the system from the graft sutered at both its ends to a blood vbessel as well as from said blood vessel, the vein filter pressed by its end to the end of said hold-down bush of the proximal head is locked by this bush, and its contents—sealed in this filter,
h) utulizing the withdrawn single-use system and checking the operation results.
